(19) United States
(12) Patent Application Publication
(10) Pub. No.: US 2006/0115439 A1
(43) Pub. Date: Jun. 1, 2006

(54) Compositions containing at least one silicone resin and glass beads

(75) Inventor: Shaoxiang Lu, Plainsboro, NJ (US)

Correspondence Address: OBLON, SPIVAK, MCCLELLAND, MAIER & NEUSTADT, P.C., 1940 DUKE STREET, ALEXANDRIA, VA, 22314, US

(73) Assignee: L'OREAL, Paris (FR)

(21) Appl. No.: 11/266,300

(22) Filed: Nov. 4, 2005

Related U.S. Application Data
(60) Provisional application No. 60/624,542, filed on Nov. 4, 2004.

(51) Int. Cl. A61K 8/89

(52) U.S. Cl. 424/63; 424/64

(57) ABSTRACT

Compositions containing at least one silicone resin and glass beads and methods of use thereof.

### FIELD OF THE INVENTION

The present invention generally relates to compositions containing at least one silicone resin and glass beads.

### BACKGROUND OF THE INVENTION

Many compositions, including pigmented cosmetic compositions (such as, for example, foundations, mascaras, lip compositions, eyeliners, etc.) and non-pigmented cosmetic compositions (such as, for example, moisturizers, sunscreens, etc.), attempt to provide longer wear and transfer resistance. Compositions which form a film after application may exhibit such properties. Generally, film forming compositions contain volatile solvents which evaporate on contact with the skin or other keratinous material, and leave behind a layer comprising waxes and/or resins, and any pigments, fillers and active agents that may be in the composition.

The use of certain silicone polymers or derivatives thereof in cosmetic compositions has also been disclosed. See e.g., U.S. Pat. Nos. 5,965,112; 5,800,816; 5,911,974; and 5,959,009, the disclosures of which are incorporated herein by reference. However, these compositions may be uncomfortable to wear as the composition that remains on the skin or other keratinous material may be a brittle or non-flexible film. These compositions may also tend to flake off the skin or other keratinous material due to poor adhesion to the material. Furthermore, prior to application, these compositions may be tacky, which may result in poor application and spreadability characteristics.

There is, therefore, a need for improved compositions such as long wearing cosmetic compositions which are comfortable to the wearer and also limit transfer to other materials such as, for example, skin or fabric, i.e., transfer resistant compositions, which also possess good cosmetic properties such as, for example, waterproof properties, and which are not tacky or “draggy” during and after application.

### SUMMARY OF THE INVENTION

The present invention relates to compositions, preferably cosmetic compositions, comprising at least one silicone resin and glass beads, as well as to methods for treating, caring for and/or making up keratinous material by applying such compositions to the keratinous material.

The present invention also relates to methods of treating, caring for and/or making up keratinous material (for example, skin) by applying compositions of the present invention to the keratinous material in an amount sufficient to treat, care for and/or make up the keratinous material.

The present invention further relates to covering or hiding skin defects associated with keratinous material (for example, skin) by applying compositions of the present invention to the keratinous material in an amount sufficient to cover or hide such skin defects.

The present invention also relates to methods of enhancing the appearance of keratinous material (for example, skin) by applying compositions of the present invention to the keratinous material in an amount sufficient to enhance the appearance of the keratinous material.

The present invention further relates to compositions having improved cosmetic properties such as, for example, improved long wear, transfer resistance or waterproof properties. The compositions may also possess improved flexibility, wearability, drying time, retention, tackiness (low), and migration over time (low).

### DETAILED DESCRIPTION OF THE INVENTION

Certain terms used herein are defined below:

“At least one” as used herein means one or more and thus includes individual components as well as mixtures/combinations.

“Film former” as used herein means a polymer that, after dissolution in at least one solvent (such as, for example, water and organic solvents), leaves a film on the substrate to which it is applied once the at least one solvent evaporates.

“Transfer resistance” as used herein refers to the quality exhibited by compositions that are not readily removed by contact with another material, such as, for example, a glass, an item of clothing or the skin, for example, when eating or drinking. Transfer resistance may be evaluated by any method known in the art for evaluating such. For example, transfer resistance of a composition may be evaluated by a “kiss” test. The “kiss” test may involve application of the composition to human lips followed by “kissing” a material, for example, a sheet of paper, after expiration of a certain amount of time following application, such as 2 minutes after application. Similarly, transfer resistance of a composition may be evaluated by the amount of product transferred from a wearer to any other substrate, such as transfer from the neck of an individual to a collar after the expiration of a certain amount of time following application. The amount of composition transferred to the substrate (e.g., collar, or paper) may then be evaluated and compared. For example, a composition may be transfer resistant if a majority of the product is left on the wearer, e.g., lips, neck, etc. In one embodiment, little or no composition is transferred to the substrate. Further, the amount transferred may be compared with that transferred by other compositions, such as commercially available compositions.

“Long wear” compositions as used herein, refers to compositions where at least one property chosen from consistency, texture, and color remains the same as at the time of application, as viewed by the naked eye, after an extended period of time, such as, for example, 1 hour, 2 hours, and further such as 8 hours. Long wear properties may be evaluated by any method known in the art for evaluating such properties. For example, long wear may be evaluated by a test involving the application of a composition to human skin (including lips) and evaluating the consistency, texture and color of the composition after an extended period of time. For example, the consistency, texture and color of a lip composition may be evaluated immediately following application and these characteristics may then be re-evaluated and compared after an individual has worn the lip composition for a certain amount of time. Further, these characteristics may be evaluated with respect to other compositions, such as commercially available compositions.

“Waterproof” as used herein refers to the ability to repel water and permanence with respect to water. Waterproof properties may be evaluated by any method known in the art for evaluating such properties. For example, a mascara composition may be applied to false eyelashes, which may then be placed in water for a certain amount of time, such as, for example, 20 minutes. Upon expiration of the pre-ascertained amount of time, the false eyelashes may be removed from the water and passed over a material, such as, for example, a sheet of paper. The extent of residue left on the material may then be evaluated and compared with other compositions, such as, for example, commercially available compositions. Similarly, for example, a composition may be applied to skin, and the skin may be submerged in water for a certain amount of time. The amount of composition remaining on the skin after the pre-ascertained amount of time may then be evaluated and compared. For example, a composition may be waterproof if a majority of the product is left on the wearer, e.g., eyelashes, skin, etc. In one embodiment, little or no composition is transferred from the wearer.

The cosmetic compositions and methods of the present invention can comprise, consist of, or consist essentially of the essential elements and limitations of the invention described herein, as well as any additional or optional ingredients, components, or limitations described herein or otherwise useful in personal care compositions intended for topical application to the skin.

Silicone resins included in the composition of the present invention can be highly cross-linked polymeric siloxane systems. The cross-linking is introduced through the incorporation of trifunctional and tetrafunctional silanes with monofunctional or difunctional, or both, silanes during manufacture of the silicone resin. As is apparent to one of ordinary skill in the art, the degree of cross-linking that is required in order to result in a silicone resin will vary according to the specific silane units incorporated into the silicone resin. Generally, silicone materials which have a sufficient level of trifunctional and tetrafunctional siloxane monomer units (and hence, a sufficient level of cross-linking) such that they dry down to a rigid, or hard, film are considered to be silicone resins. The ratio of oxygen atoms to silicon atoms is indicative of the level of cross-linking in a particular silicone material. Silicone resins suitable for use in the compositions of the present invention generally have at least about 1.1 oxygen atoms per silicon atom. Preferably, the ratio of oxygen to silicon atoms is at least about 1.2:1.0. Silanes used in the manufacture of silicone resins include, but are not limited to: monomethyl-, dimethyl-, trimethyl-, monophenyl-, diphenyl-, methylphenyl-, monovinyl-, and methylvinyl-chlorosilanes, and tetra-chlorosilane, with the methyl-substituted silanes being most commonly utilized. Preferred resins are available from General Electric as GE SS4230 and GE SS4267.

Commercially available silicone resins are generally supplied in a dissolved form in a low viscosity volatile or non-volatile silicone fluid. The at leas one silicone resin for use herein should be supplied and incorporated into the present compositions in such dissolved form, as will be readily apparent to one of ordinary skill in the art.

Silicone materials and silicone resins in particular, can conveniently be identified according to a shorthand nomenclature system known to those of ordinary skill in the art as “MDTQ” nomenclature. Under this system, the silicone is described according to presence of various siloxane monomer units which make up the silicone. Briefly, the symbol M denotes the monofunctional unit (CH₃)₃SiO_{0.5}; D denotes the difunctional unit (CH₃)₂SiO; T denotes the trifunctional unit (CH₃)SiO_{1.5}; and Q denotes the quadra- or tetra-functional unit SiO₂. Primes of the unit symbols (e.g. M′, D′, T′, and Q′) denote substituents other than methyl, and must be specifically defined for each occurrence. Typical alternate substituents include, but are not limited to, groups such as vinyl, phenyls, amines, hydroxyls, and the like. The molar ratios of the various units, either in terms of subscripts to the symbols indicating the total number of each type of unit in the silicone (or an average thereof) or as specifically indicated ratios in combination with molecular weight complete the description of the silicone material under the MDTQ system. Higher relative molar amounts of T, Q, T′ and/or Q′ to D, D′, M and/or M′ in a silicone resin indicates higher levels of cross-linking. As discussed above, however, the overall level of cross-linking can also be indicated by the oxygen to silicon ratio.

Preferred silicone resins for use in the compositions of the present invention include, but are not limited to MQ, MT, MTQ, MDT and MDTQ resins. Methyl is a preferred silicone substituent. Especially preferred silicone resins are MQ resins, wherein the M:Q ratio is from about 0.5:1.0 to about 1.5:1.0 and the average molecular weight of the silicone resin is from about 1000 to about 10,000.

According to one embodiment of the present invention, the silicone resin is a polymethylsilsesquioxane film former (film forming agent). In this regard, it is noted that not all polymethylsilsesquioxanes are film formers and, thus, not all polymethylsilsesquioxanes are encompassed within the scope of the present invention. For example, highly polymerized polymethylsilsesquioxanes (T Resins), such as Tospearl™ from Toshiba and KMP590 from Shin-Etsu, are highly insoluble non-film formers and, thus, not within the scope of the present invention. The molecular weights of such polymethylsilsesquioxanes are difficult to determine; however, they generally contain at least 1000 T units.

According to preferred embodiments, the number of T units of the at least one polymethylsilsesquioxane film former is less than or equal to 500, more preferably ranging from about 50 to about 500. Moreover, it is preferred that the at least one film forming agent possess at least one of the following characteristics: (1) it has a softening point ranging from about 40° C. to about 80° C.; (2) it is soluble in solvents chosen from volatile silicones and organic solvents; (3) it has a weight-average molecular weight ranging from about 500 to about 50,000, more preferably from about 500 to about 20,000 and most preferably about 10,000; and (4) it does not have a substituent replacing the methyl groups of the T units. A non-limiting example of an acceptable polymethylsilsesquioxane film former is Belsil PMS MK, also referred to as Resin MK, available from Wacker Chemie. This polymethylsilsesquioxane film former is a polymer comprising polymerized repeating units of CH₃SiO_{3/2} (T units) and may also contain up to 1% by weight or by mole of units of the formula (CH₃)₂SiO_{2/2} (D units). The weight-average molecular weight of this polymer has been estimated to be 10,000. It is believed that the polymers are in a “cage” and “ladder” configuration. The weight-average molecular weight of the “cage” unit has been calculated to be 536 g/mol. The majority of the polymer is in the “ladder” configuration, wherein the ends of the polymer are capped with ethoxy (CH₃CH₂O) groups. The ethoxy groups are generally present in an amount of 4.5% by weight and the mole percent is generally 7% (silicone units). As ethoxy groups may react with water, a small and variable amount of SiOH may also be present in the polymer.

Another non-limiting example of the at least one polymethylsilsesquioxane film former suitable for use in the present invention is KR-220L, which is available from SHIN-ETSU. This polymethylsilsesquioxane film former is composed of silicone T-units (i.e, those of formula CH₃SiO_{3/2}) and has Si—OH (or silanol) end units. There are no D units in KR-220L.

Other non-limiting examples of acceptable polymethylsilsesquioxane film formers that may be useful in the present invention include KR-242A (which is comprised of methyl T units (98%) and dimethyl D units (2%) and has Si—OH end units) and KR-251 (which is comprised of methyl T units (88%) and dimethyl D units (12%) and has Si—OH end units), both of which are available from SHIN-ETSU.

The silicone resin may be an MQ resin. In one embodiment of the present invention, there can be at least two different foreseeable silicone resins. In one embodiment, on resin can be a polysilsesquioxane film former and another resin an MQ resin.

Non-limiting examples of acceptable MQ resins are described in U.S. Pat. No. 5,330,747, which is incorporated herein by reference in its entirety. Preferably, the MQ resin has an M:Q ratio of about 0.5 and an n value of about 1.5. Most preferably, the MQ resin is trimethylsiloxysilicate. Non-limiting examples of acceptable commercially available MQ resins can be obtained from Wacker Silicones Corporation of Adrian Mich. (e.g., Wacker 803 and 804) and the General Electric Company (e.g., G. E. 1170-002 and SR 1000).

Depending on the composition and/or its intended use, the concentration of the at least one silicone resin film former may vary. One skilled in the art will be able to determine routinely the amount of the at least one silicone resin to be included in a composition depending on the desired application. Preferably, the at least one silicone resin is present in an amount effective to provide long wear and/or transfer resistance. For example, for cosmetic foundations, the at least one silicone resin may be present in the composition in an amount generally ranging from about 0.1% to about 90% by weight relative to the total weight of the composition, including 1, 3, 5, 10, 15, 20, 25, 30, 35, 40, 45, 50, 55, 60, 65, 70, 75, 80, 85% by weight relative to the total weight of the composition as well as all values and ranges there between. For eyeliner compositions, the at least one silicone resin may be present in an amount generally ranging from about 5% to about 90% by weight relative to the total weight of the composition including 10, 15, 20, 25, 30, 35, 40, 45, 50, 55, 60, 65, 70, 75, 80, 85% by weight relative to the total weight of the composition and all values and subranges there between. For lip compositions, such as lipstick or liquid lip color products, the at least one silicone resin may be present in an amount generally ranging from about 1% to about 90% by weight relative to the total weight of the composition, including 5, 10, 15, 20, 25, 30, 35, 40, 45, 50, 55, 60, 65, 70, 75, 80, 85% by weight relative to the total weight of the composition and all values and subranges there between. For mascara compositions, the at least one silicone resin may be present in an amount generally ranging from about 1% to about 50% by weight relative to the total weight of the composition, including 10, 15, 20, 25, 30, 35, 40, 45% by weight relative to the total weight of the composition and all values and subranges there between.

In one embodiment, polsilsesquioxane film former may be used together with an MQ resin. If used together, the polysilsesquioxane film former and the MQ resin constitute a high substantivity film forming material. Preferably, the aggregate total of the at least one polysilsesquioxane film former and the at least one MQ resin is from about 0.1% to about 60% by weight relative to the total weight of the composition, preferably from about 3% to about 40% by weight relative to the total weight of the composition, more preferably from about 5% to about 20% by weight relative to the total weight of the composition, including 10, 15, 20, 25, 30, 35, 40, 45, 50, 55% by weight relative to the total weight of the composition and all values and subranges there between.

Together, the polysilsesquioxane film former and the MQ resin constitute a high substantivity film forming material. Preferably, the aggregate total of the at least one polysilsesquioxane film former and the at least one MQ resin is from about 0.1% to about 60% by weight relative to the total weight of the composition, preferably from about 3% to about 40% by weight relative to the total weight of the composition, more preferably from about 5% to about 20% by weight relative to the total weight of the composition, including 1, 3, 5, 10, 15, 20, 25, 30, 35, 40, 45, 50, 55% by weight relative to the total weight of the composition and all values and subranges there between.

According to the one aspect of the present invention, compositions comprising the silicone resins further comprise glass beads, which can be all of one type of glass bead or mixtures of glass beads. The glass beads employed are applicable for use in cosmetic applications, for example, glass beads made of borosilicates such as calcium borosilicate, calcium aluminum borosilicate, and/or sodium borosilicate. In a further embodiment, the glass beads can be functionalized with various groups such as, coated or uncoated, coated or uncoated surface treated glass beads Acceptable glass beads can be solid or hollow, colorless or colored, and coated or uncoated. Moreover, the glass beads can be of any shape. Preferably, the glass beads have a substantially spherical or ovoid shape which can provide the composition with a softer feel upon application as well as allow more thorough distribution of the particles throughout the composition.

According to one embodiment, the glass beads (or microspheres) are essentially homogenous and essentially uniform in sphericity.

Preferably, the glass beads have an average size ranging from about 0.5 μm to about 300 μm, more preferably from about 1 μm to about 200 μm, and most preferably from about 5 μm to about 50 μm. Generally speaking, the smaller the size of the beads, the greater the staying power of the beads and, thus, the greater the transfer-resistance of the composition.

Depending on the composition and/or its intended use, the concentration of glass beads may vary. One skilled in the art can determine routinely the amount of the glass beads to be included in a composition depending on the desired application. According to one embodiment, the glass beads are present in an amount sufficient to increase or improve the transfer-resistance of the composition. Preferably, the glass beads are present in an amount ranging from about 0.1% to about 50% by weight relative to the total weight of the composition, including 1, 3, 5, 10, 15, 20, 25, 30, 35, 40, 45% by weight relative to the total weight of the composition and all values and subranges there between, and preferably from about 1% to about 25% by weight relative to the total weight of the composition, more preferably from about 2% to about 10% by weight relative to the total weight of the composition.

Examples of cosmetic glass microspheres according to this invention include those amorphous hollow microspheres of calcium aluminum borosilicate marketed by Presperse Inc. under the trademark LUXSIL®. It is a high slip, chemically inert, non-absorbent, non-porous free-flowing white powder, essentially uniformly spherical in form, having a mean particle size ranging from 9-13 microns.

The typical particle size distribution thereof is as follows:

| | |
|---|---|
| 10% | 3-6 μm |
| 50% | 8-12 μm |
| 90% | 16-23 μm |

Moreover, typical density ranges from 1.05-1.15 g/cc, has a weight loss on drying of less than 0.5%, and contains less than 20 ppm of lead and less than 3 ppm of arsenic. Its CAS No. is 65997-17-3.

Preferred concentrations of the LUXSIL® free-flowing, spherical white powder microbeads is on the order of 0.1% to 10% w/w in the formulation. Concentrations of from 1% to 5% are even more preferred.

Other suitable glass microspheres according to the present invention, whether hollow, solid or otherwise, include those sodium borosilicate particulates marketed by PQ Corporation under the trademark Q-CEL 570, having a granulometry on the order of 20 μm. Also suitable are those calcium/sodium borosilicate hollow microspheres marketed by 3M under the trademarks ES 22 and 1K, having a granulometry up to 70 μm, and 3M's Scotchlite K20 product, calcium/sodium borosilicate microspheres having a mean particle size on the order of 10 μm. Further examples of glass beads (or microspheres) which can be used in the present invention include those described in WO03/045345, the relevant contents of which are incorporated herein by reference.

According to preferred embodiments of the present invention, compositions containing the at least one silicone resin, glass beads, and pigments are provided. Preferably, the pigments are present in an amount ranging from about 0.1% to about 30% by weight relative to the total weight of the composition, more preferably from about 1% to about 30% by weight relative to the total weight of the composition, and most preferably from about 5% to about 30% by weight relative to the total weight of the composition. Acceptable pigments include any organic or inorganic color or pigment suitable for topical application to the skin. Preferably, the pigment is a titanium oxide, iron oxide, chalk, talc, titanated micas, and combinations thereof. Non-limiting examples of acceptable pigments include titanium dioxide, D & C Red No. 7 Calcium Lake, D & C Red No. 21 Aluminum Lake, Iron Oxides, FD & C Yellow No. 5 Aluminum Lake, FD & C Blue No. 1 Aluminum Lake and any other pigments (including treated pigments) known in the cosmetic arts.

According to other preferred embodiments of the present invention, anhydrous compositions comprising at least one silicone resin, glass beads, and pigment are provided. By “anhydrous,” it is meant that virtually no water exists in the composition, although it is understood that minimal amounts of water may exist (for example, up to 1% by weight relative to the total weight of the composition). In some embodiments, the anhydrous pigmented composition is in the form of a solid stick such as, for example, a lipstick or liquid lip color products or a stick foundation, a paste or a liquid.

According to another preferred embodiment of the present invention, compositions comprising the silicone resin, glass beads and water are provided. Preferably, these compositions further comprise pigments. Where the compositions contain pigments, the compositions are preferably lip compositions such as, for example, a lipstick or liquid lip color products, a foundation or a mascara. Where the compositions do not contain pigments, the compositions are preferably a cleansing, skin care, hair care or sun care compositions such as, for example, a shampoo, a moisturizer, a sunless tanner, or a sunscreen composition.

According to this embodiment of the present invention, water is present from about 1% to about 90% by weight relative to the total weight of the composition, preferably from about 10% to about 60% by weight relative to the total weight of the composition, and most preferably from about 30% to about 50% by weight relative to the total weight of the composition.

The compositions of the present invention may provide at least one property chosen from long wear and transfer resistance in a broad range of applications. These applications include, for example, pigmented cosmetics (including foundations, concealers, mascaras, eyeliners, lipsticks, liquid lip color products, eyeshadows); hair compositions (including hair sprays, gels and mousses); sunscreen compositions; skin care and/or treatment compositions (including lotions, such as moisturizing lotions, lotions with active ingredients, and lotions with fragrances). Moreover, the compositions may also be effective in waterproofing. For example, the compositions may impede dehydration of the substrate to which they are applied (for example, skin) by forming an occlusive film and reducing transepidermal water loss. Furthermore, the compositions may provide a barrier between the substrate to which they are applied and the environment, thereby entrapping any active and/or functional ingredients there between. This barrier may boost the activity of any active and/or functional ingredients (such as, for example, the SPF and UV light protection of sunscreen ingredients or the anti-signs of aging (for example, wrinkles) activity of compounds such as retinoids and alpha-hydroxy acids) and may block the effect of humidity and the environment on such ingredients.

The compositions of the present invention may be in any product form. For example, the compositions may be make-up products such as lip compositions (including lipstick basecoats and topcoats), lip pencils, foundations (including foundations which may be cast in the form of a stick or a dish), blushers, concealer products, temporary tattoo products, eyeliners, and mascaras. The compositions may also be skin care products and/or products for treating at least one keratinous material such as, for example, sunscreens, after-sun products, sunless tanning products, hair care products (including shampoos and conditioners), moisturizers, anti-aging products, a deodorant etc.

Furthermore, the compositions of the present invention may be in any physical form. For example, the compositions may be in the form of a paste, a solid, a gel, or a cream. The composition may be a simple emulsion, such as an oil-in-water or water-in-oil emulsion, a multiple emulsion, such as an oil-in-water-in-oil emulsion or a water-in-oil-in-water emulsion, or a solid, rigid or supple gel, including anhydrous gels. The composition may also be in a form chosen from a translucent gel and a transparent gel. The composition can also be a molded composition or cast as a stick or a dish. The composition may also be a solid such as a molded stick or a poured stick, which may or may not be anhydrous. Alternatively, the composition may be liquid such as, for example, a liquid lip basecoat or topcoat which contains minimal amounts of wax or no wax at all.

Depending on the intended application and/or physical form, such as a stick, hardness of the composition may also be considered. The hardness of a composition may, for example, be expressed in grams (g). The composition of the present invention may, for example, have a hardness ranging from 20 g to 2000 g, such as from 20 g to 900 g, and further such as from 20 g to 600 g.

This hardness is measured in one of two ways. A first test for hardness is according to a method of penetrating a probe into said composition and in particular using a texture analyzer (for example TA-XT2 from Rhéo) equipped with an ebonite cylinder of height 25 mm and diameter 8 mm. The hardness measurement is carried out at 20° C. at the center of 5 samples of said composition. The cylinder is introduced into each sample of composition at a pre-speed of 2 mm/s and then at a speed of 0.5 mm/s and finally at a post-speed of 2 mm/s, the total displacement being 1 mm. The recorded hardness value is that of the maximum peak observed. The measurement error is ±50 g.

The second test for hardness is the “cheese wire” method, which involves cutting an 8.1 mm tube of composition and measuring its hardness at 20° C. using a DFGHS 2 tensile testing machine from Indelco-Chatillon Co. at a speed of 100 mm/minute. The hardness value from this method is expressed in grams as the shear force required to cut a stick under the above conditions. According to this method, the hardness of compositions according to the present invention which may be in stick form may, for example, range from 30 g to 300 g, such as from 30 g to 250 g, and further such as from 30 g to 200 g.

The hardness of the composition of the present invention may be such that the compositions are self-supporting and can easily disintegrate to form a satisfactory deposit on a keratinous material. In addition, this hardness may impart good impact strength to the inventive compositions which may be molded or cast, for example, in stick or dish form.

The skilled artisan may choose to evaluate a composition using at least one of the tests for hardness outlined above based on the application envisaged and the hardness desired. If one obtains an acceptable hardness value, in view of the intended application, from at least one of these hardness tests, the composition falls within the scope of the invention. According to the present invention, the compositions in stick form may also possess the properties of deformable, flexible elastic solids and may also have noteworthy elastic softness upon application to a keratinous material.

The compositions of the present invention may further comprise at least one additional ingredient. Non-limiting examples of the at least one additional ingredient include gelling agents, oils, waxes, preservatives, suspending agents, thickening agents, solvents, surfactants, emollients, film formers, spreading agent, dispersants, antifoaming agents, wetting agents, UV-screening agents, antioxidants, perfumes, fatty acids, fillers, active agents, moisturizers, vitamins, biological materials, and derivatives of any of the foregoing.

Characteristics of the at least one additional ingredient may include the ability to impart an oily feeling and/or increased spreadability, for example, as observed with some esters and organic sunscreens.

Acceptable thickeners include, for example, those derived from or otherwise similar to inorganic clays, preferably montmorillonite clays. Montmorillonite clays are those which contain the mineral montmorillonite and are characterized by having a suspending lattice. Examples of montmorillonite clays suitable for use herein include the bentonites, hectorites, colloidal magnesium aluminum silicates, and mixtures thereof.

Highly preferred inorganic thickening agents include hydrophobically treated montmorillonite clays, including hydrophobic bentonites available from Rheox, Inc., Hightstown, N.J., U.S.A., under the tradename Bentone®. Non limiting examples of suitable Bentone® materials for use herein include Bentone® 27, Bentone® 34, Bentone® 38, Bentone® EW, Bentone® Gel CAO, Bentone® Gel IPM Bentone® Gel ISD, Bentone® Gel LOI, Bentone® Gel MIO, Bentone® Gel MIO, Bentone® Gel MIO A-40, and many other similar materials. Most preferred is Bentone®. Gels that contain hydrophobically treated bentonite clays in the form of quaternium-18 hectorite.

An appropriate concentration of the thickener, if present, may be determined by one of skill in the art and can vary considerably based on the application. For example, for cosmetic compositions, the thickener may be used in an amount from 0.1% to 20% such as, for example, from 1% to 10% by weight, relative to the total weight of the composition.

The additional film former may be any material with film forming properties. Acceptable film formers include, for example, those listed at pages 1744 to 1747 of the CTFA International Cosmetic Ingredient Dictionary, 8^{th} edition (2000). Specifically, the additional film forming agent may be chosen from di-block copolymer film formers, tri-block copolymer film formers, multi-block copolymer film formers, radial copolymer film formers, and star block copolymer film formers. As defined herein, the foregoing copolymers comprise distinctive arrangements of both hard and soft segments. A non-limiting example of a hard segment is styrene, while non-limiting examples of soft segments include ethylene, propylene, butylene and combinations of any of the foregoing soft segments.

Non-limiting examples of acceptable additional film forming agents include: vinylpyrrolidone/vinyl acetate (PVP/VA) copolymers, such as Luviskol® VA copolymers available from BASF® Corporation and PVP/VA series copolymers available from from ISP; acrylic fluorinated emulsion film formers, such as Foraperle® film formers (e.g., Foraperle® 303 D available from Elf Atochem), although Foraperle® may not be suitable for some cosmetic formulations; GANEX® copolymers, such as butylated PVP, PVP/Hexadecene copolymers, PVP/Eicosene copolymers, and tricontanyl; Poly(vinylpyrrolidone/diethylaminoethyl methacrylate) copolymers and PVP/Dimethylaminoethylmethacrylate copolymers such as Copolymer 845 available from I.S.P.; Resin ACO-5014 (Imidized IB/MA copolymer); other PVP based polymers and copolymers; alkyl cycloalkylacrylate copolymers, such as those disclosed in WO 98/42298, the disclosure of which is hereby incorporated by reference; Mexomere® film formers and other allyl stearate/vinyl acetate copolymers; polyolprepolymers, such as PPG-12/SMDI copolymer, also called Poly(oxy-1,2-ethanediyl), α-hydro-ω-hydroxy-polymer with 1,1′-methylene-bis-(4-isocyanatocyclohexane) available from Barnet; and Avalure™ AC Polymers (Acrylates Copolymer) and Avalure™ UR polymers (Polyurethane Dispersions), available from BFGoodrich.

The additional film forming agent may also be Kraton® rubber copolymers (which are available from Shell Chemical Company) or similar gelling agents. The Kraton® rubber configuration is well known in the art and any block copolymer film former, such as gelling agents, with a similar configuration is within the practice of the invention. For example, the additional film forming agent may be Kraton® rubber copolymers which is present in a gel in an amount ranging from 10% to 20% by weight relative to the total weight of the composition.

Each molecule of Kraton® rubber comprises block segments of (i) styrene units and (ii) rubber monomer and/or co-monomer units. Thus, Kraton® rubber copolymers are thermoplastic elastomers in which the polymer chains have a configuration chosen from tri-block configurations, di-block configurations, radial block configurations, star block configurations and mixtures of any of the foregoing configurations. The configurations of each of the Kraton® rubbers form separate polystyrene and rubber domains.

The Kraton® tri-block rubber copolymers are comprised of polystyrene segments on each end of a rubber segment, while the Kraton® di-block rubber copolymers are comprised of a polystyrene segment attached to a rubber segment. The most common Kraton® triblock copolymers are linear A-B-A block type styrene-butadiene-styrene copolymers, linear A-B-A block styrene-isoprene-styrene copolymers, and linear A-B-A block styrene-ethylenebutylene-styrene copolymers. Non-limiting examples of the Kraton® di-block copolymers include AB block type copolymers such as styrene-ethylenepropylene copolymers, styrene-ethylenebutylene copolymers, styrene-butadiene copolymers, and styrene-isoprene copolymers. The Kraton® radial or star configuration, in another embodiment, may, for example, be a four-point or other multipoint star made of rubber with a polystyrene segment attached to each end of a rubber segment.

The additional film forming agent may also be chosen from block copolymer film formers comprising at least one styrene/butylene/ethylene/styrene copolymer (tri-block) and at least one ethylene/propylene/styrene copolymer (radial or star block). Similarly, corresponding block copolymers which are referred to as “hydrogenated” block copolymers by the manufacturer (e.g., hydrogenated styrene/butylene/ethylene/styrene copolymer (tri-block) and hydrogenated ethylene/propylene/styrene copolymer (radial or star block)) are within the scope of the invention. Non-limiting examples of suitable block copolymer film formers include Versagel M5960 and Versagel M5970, which are available from Penreco of Houston Tex., and block copolymers available from Brooks Industries, such as Gel Base.

An appropriate concentration of the film former, if present, may be determined by one of skill in the art and can vary considerably based on the application. For example, for cosmetic compositions, the film former may be used in an amount from 0.1% to 20% such as, for example, from 1% to 10% by weight, relative to the total weight of the composition.

The compositions of the present invention may also contain one or more volatile oils or solvents. For the purposes of the invention, the expression “volatile solvent or oil” means any non-aqueous medium capable of evaporating on contact with the skin or the lips in less than one hour at room temperature and atmospheric pressure. The volatile solvent(s) of the invention is(are) organic solvents, such as volatile cosmetic oils that are liquid at room temperature, having a non-zero vapor pressure, at room temperature and atmospheric pressure, ranging in particular from 10⁻² to 300 mmHg and, for example, greater than 0.3 mmHg. The expression “non-volatile oil” means an oil which remains on the skin or the lips at room temperature and atmospheric pressure for at least several hours, such as those having a vapor pressure of less than 10⁻² mmHg.

The volatile solvents, if present, may facilitate the staying power or long wearing properties of the composition on the skin, the lips or superficial body growths. The volatile liquid carrier for use in the liquid cosmetic compositions of the present invention include volatile hydrocarbons, volatile silicones and combinations thereof, the concentration of which within the liquid cosmetic compositions generally ranges from about 10% to about 90%, preferably from about 20% to about 80%, and more preferably from about 40% to about 60%, by weight of the compositions.

Volatile hydrocarbons suitable for use as a volatile oil include those hydrocarbons having boiling points in the range of from about 60° C. to about 260° C., more preferably volatile hydrocarbons having from about C₈ to about C₂₀ chain lengths, more preferably C₈ to C₂₀ isoparaffins. Preferred isoparaffins for use herein include isododecane, isohexadecane, isoeocosane, 2,2,4-trimethylpentane, 2,3-dimethylhexane and combinations thereof. Most preferred is isododecane.

Volatile silicones suitable for use as a volatile liquid carrier in the liquid cosmetic compositions herein include those volatile silicones as described in Todd et al., “Volatile Silicone Fluids for Cosmetics”, Cosmetics and Toiletries, 91:27-32 (1976), which descriptions are incorporated herein by reference. The volatile silicone can be linear, cyclic or branched, but is preferably a cyclic silicone having from about 3 to about 7, more preferably from about 4 to about 5, silicon atoms. Most preferably are those which conform to the formula: 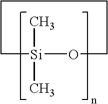
wherein n is from about 3 to about 7, preferably from about 4 to about 5, most preferably 5. Suitable volatile silicones for use herein include, but are not limited to, Cyclomethicone D-5 (commercially available from G. E. Silicones); Dow Corning 344, and Dow Corning 345 (commercially available from Dow Corning Corp.); GE 7207, GE 7158 and Silicone Fluids SF-1202 and SF-1173 (available from General Electric Co.); SWS-03314, SWS-03400, F-222, F-223, F-250, F-251 (available from SWS Silicones Corp.); Volatile Silicones 7158, 7207, 7349 (available from Union Carbide); Masil SF-V (available from Mazer) and combinations thereof. Cyclopentasiloxane is most preferred among the volatile silicone liquids.

The compositions of the present invention may also contain one or more sun screening agents. Acceptable screening agents may be chemical absorbers which actually absorb harmful ultraviolet radiation. It is well known that chemical absorbers are classified, depending on the type of radiation they protect against, as either UV-A or UV-B absorbers. UV-A absorbers generally absorb radiation in the 320 to 400 nm region of the ultraviolet spectrum. UV-A absorbers include anthranilates, benzophenones, and dibenzoyl methanes. UV-B absorbers generally absorb radiation in the 280 to 320 nm region of the ultraviolet spectrum. UV-B absorbers include p-aminobenzoic acid derivatives, camphor derivatives, cinnamates, and salicylates.

Classifying the chemical absorbers generally as UV-A or UV-B absorbers is accepted within the industry. However, a more precise classification is one based upon the chemical properties of the sunscreens. There are eight major classifications of sunscreen chemical properties which are discussed at length in “Sunscreens—Development, Evaluation and Regulatory Aspects,” by N. Shaath et al., 2nd. Edition, pages 269-273, Marcel Dekker, Inc. (1997). This discussion, in its entirety, is incorporated by reference herein.

The sunscreen compositions which may be formulated according to the present invention typically comprise chemical absorbers, but may also comprise physical blockers. Exemplary sunscreens which may be formulated into the compositions of the present invention are chemical absorbers such as p-aminobenzoic acid derivatives, anthranilates, benzophenones, camphor derivatives, cinnamic derivatives, dibenzoyl methanes (such as avobenzone also known as Parsol® 1789), diphenylacrylate derivatives, salicylic derivatives, triazine derivatives, benzimidazole compounds, bis-benzoazolyl derivatives, methylene bis-(hydroxyphenylbenzotriazole) compounds, the sunscreen polymers and silicones, or mixtures thereof. These are variously described in U.S. Pat. Nos. 2,463,264, 4,367,390, 5,166,355 and 5,237,071 and in EP-0,863,145, EP-0,517,104, EP-0,570,838, EP-0,796,851, EP-0,775,698, EP-0,878,469, EP-0,933,376, EP-0,893,119, EP-0,669,323, GB-2,303,549, DE-1,972,184 and WO-93/04665, also expressly incorporated by reference. Also exemplary of the sunscreens which may be formulated into the compositions of this invention are physical blockers such as cerium oxides, chromium oxides, cobalt oxides, iron oxides, red petrolatum, silicone-treated titanium dioxide, titanium dioxide, zinc oxide, and/or zirconium oxide, or mixtures thereof.

A wide variety of sunscreens is described in U.S. Pat. No. 5,087,445, issued to Haffey et al. on Feb. 11, 1992; U.S. Pat. No. 5,073,372, issued to Turner et al. on Dec. 17, 1991; and Chapter VIII of Cosmetics and Science and Technology by Segarin et al., pages 189 et seq. (1957), all of which are incorporated herein by reference in their entirety.

Sunscreens which may be formulated into the compositions of the instant invention are those selected from among: aminobenzoic acid, amyldimethyl PABA, cinoxate, diethanolamine p-methoxycinnamate, digalloyl trioleate, dioxybenzone, 2-ethoxyethyl p-methoxycinnamate, ethyl 4-bis(hydroxypropyl)aminobenzoate, 2-ethylhexyl-2-cyano-3,3-diphenylacrylate, ethylhexyl p-methoxycinnamate, 2-ethylhexyl salicylate, glyceryl aminobenzoate, homomenthyl salicylate, homosalate, 3-imidazol-4-ylacrylic acid and ethyl ester, methyl anthranilate, octyldimethyl PABA, 2-phenylbenzimidazole-5-sulfonic acid and salts, red petrolatum, sulisobenzone, titanium dioxide, triethanolamine salicylate, N,N,N-trimethyl-4-(2-oxoborn-3-ylidene methyl)anillinium methyl sulfate, and mixtures thereof.

Sunscreens active in the UV-A and/or UV-B range can also include: p-aminobenzoic acid, oxyethylene (25 mol) p-aminobenzoate, 2-ethylhexyl p-dimethylaminobenzoate, ethyl N-oxypropylene p-aminobenzoate, glycerol p-aminobenzoate, 4-isopropylbenzyl salicylate, 2-ethylhexyl 4-methoxycinnamate, methyl diisopropylcinnamate, isoamyl 4-methoxycinnamate, diethanolamine 4-methoxycinnamate, 3-(4′-trimethylammunium)-benzyliden-bornan-2-one methylsulfate, 2-hydroxy-4-methoxybenzophenone, 2-hydroxy-4-methoxybenzophenone-5-sulfonate, 2,4-dihydroxybenzophenone, 2,2′,4,4′-tetrahydroxybenzophenone, 2,2′-dihydroxy-4,4′dimethoxybenzophenone, 2-hydroxy-4-n-octoxybenzophenone, 2-hydroxy-4-methoxy-4′-methoxybenzophenone, -(2-oxoborn-3-ylidene)-tolyl-4-sulfonic acid and soluble salts thereof, 3-(4′-sulfo)benzyliden-bornan-2-one and soluble salts thereof, 3-(4′methylbenzylidene)-d,l-camphor, 3-benzylidene-d,l-camphor, benzene 1,4-di(3-methylidene-10-camphosulfonic) acid and salts thereof (the product Mexoryl SX described in U.S. Pat. No. 4,585,597 issued to Lange et al. on Apr. 29, 1986), urocanic acid, 2,4,6-tris[p-(2′-ethylhexyl-1′-oxycarbonyl)-anilino]-1,3,5-triazine, 2-[(p-(tertiobutylamido)anilino]-4,6-bis-[(p-(2′-ethylhexyl-1′-oxycarbonyl)anilino]-1,3,5-triazine, 2,4-bis {[4-(2-ethyl-hexyloxy)]-2-hydroxyl-phenyl}-6-(4-methoxy-phenyl)-1,3,5-triazine (“TINOSORB S” marketed by Ciba), the polymer of N-(2 et 4)-[(2-oxoborn-3-yliden)methyl]benzyl]-acrylamide, 1,4-bisbenzimidazolyl-phenylen-3,3′,5,5′-tetrasulfonic acid and salts thereof, the benzalmalonate-substituted polyorganosiloxanes, the benzotriazole-substituted polyorganosiloxanes (Drometrizole Trisiloxane), dispersed 2,2′-methylene-bis-[6-(2H-benzotriazol-2-yl)-4-(1,1,3,3-tetramethylbutyl)phenol] such as that marketed under the trademark MIXXIM BB/100 by Fairmount Chemical, or micronized in dispersed form thereof such as that marketed under the trademark TINOSORB M by Ciba-Geigy, and solubilized 2,2′-methylene-bis-[6-(2H-benzotriazol-2-yl)-4-(methyl)phenol] such as that marketed under the trademark MIXXIM BB/200 by Fairmount Chemical. Typically combinations of one of more of these sunscreens are used.

The dibenzoyl methane derivatives other than avobenzone are described, for example, in FR-2,326,405, FR-2,440,933 and EP-0,114,607, hereby expressly incorporated by reference.

Other dibenzoyl methane sunscreens other than avobenzone include (whether singly or in any combination): 2-methyldibenzoylmethane; 4-methyldibenzoylmethane; 4-isopropyldibenzoylmethane; 4-tert.-butyldibenzoylmethane; 2,4-dimethyldibenzoylmethane; 2,5-dimethyldibenzoylmethane; 4,4′-diisopropyldibenzoylmethane; 4,4′-dimethoxydibenzoylmethane; 2-methyl-5-isopropyl-4′-methoxydibenzoylmethane; 2-methyl-5-tert.-butyl-4′-methoxydibenzoylmethane; 2,4-dimethyl-4′-methoxydibenzoylmethane; 2,6-dimethyl-4-tert.-butyl-4′-methoxydibenzoylmethane; Additional sunscreens that can be used are described in pages 2954-2955 of the International Cosmetic Ingredient Dictionary and Handbook (9^{th} ed. 2002).

The compositions of the invention may further comprise at least one additional fatty material. The compositions of the invention may further include formulation aids which are usually employed in the field of application envisaged. The formulation aids used in the present invention can be, but are not limited to, fatty substances. Useful fatty substances include, but are not limited to, organic and organosilicone emulsifiers. Examples of acceptable organic emulsifiers include any ethoxylated surfactant known in the art such as Polysorbate-20, Laureth-7, Laureth-4, Sepigel® 305 available from SEPPIC and other similar ingredients disclosed in the International Cosmetic Ingredient Dictionary and Handbook Vol. 4 (9^{th} ed. 2002), more particularly the emulsifiers disclosed on pages 2962-2971. The disclosure of the International Cosmetic Ingredient Dictionary and Handbook Vol. 4, pages 2962-2971, is hereby incorporated by reference. Examples of organosilicone emulsifiers include cetyl dimethicone copolyol-polyglyceryl-4-isostearate-hexylaurate (ABIL® WE 09) available from Goldschmidt Chemical Corporation, Cetyl Dimethicone Copolyol (ABIL® EM 90), (ABIL® EM 97), Laurylmethicone Copolyol (5200), Cyclomethicone (and) Dimethicone Copolyol (DC 5225 C and DC 3225 C) available from GE Silicones, Cyclopentasiloxane & Dimethicone Copolyol (GE SF 1528) or any other formulation aids known by one of skill in the art. Other fatty substances useful as formulation aids include but are not limited to oils such as vegetable and mineral oil; waxes of natural origin, such as beeswax, carnauba wax, candelilla wax, ouricury wax, Japan wax, cork fiber wax, sugar cane wax, paraffin waxes, lignite wax, microcrystalline waxes, lanolin wax, montan wax and ozokerites, hydrogenated oils such as hydrogenated jojoba oil, jojoba esters; waxes of synthetic origin, such as polyethylene waxes derived from polymerization of ethylene, waxes obtained by Fischer-Tropsch synthesis, fatty acid esters and glycerides, and derivatives thereof; and hydrocarbon gels or bentone type gels, such as Gel SS71, Gel EA2786, Quaternium-18 Bentonite, 38 CE, Gel ISD V or Gel ISD. In one embodiment, the wax is a hydroxy polyester wax derived from the natural fatty acids and synthetic fatty alcohols yielding a wax that mimics the complex polyester fraction of natural beeswax which gives beeswax its unique plastic properties, for example Kester K82P (Koster-Keunan, Inc.) which is made of a range of hydroxyl polyesters with an average chain length of 66 carbon atoms, C20-40 alkyl hydroxystearyl stearate (CAS 151661-95-9).

These substances may be selected variously by the person skilled in the art in order to prepare a composition which has the desired properties, for example, consistency or texture.

Plasticizers may also be added to the compositions to improve the flexibility and cosmetic properties of the resulting formulation. Plasticizers are materials which soften synthetic polymers. They are frequently required to avoid brittleness and cracking of film formers. One skilled in the art may routinely vary the amount of plasticizer desired based on the properties desired and the application envisaged. Plasticizers useful in the practice of the invention include lecithin, polysorbates, dimethicone copolyol, glycols, citrate esters, glycerin, dimethicone, and other similar ingredients disclosed in the International Cosmetic Ingredient Dictionary and Handbook Vol. 4 (9^{th} ed. 2002), more particularly the plasticizers disclosed on page 2927. The disclosure of the International Cosmetic Ingredient Dictionary and Handbook Vol. 4, page 2927, is hereby incorporated by reference.

According to preferred embodiments of the present invention, methods of treating, caring for and/or making up keratinous material such as skin, lips, hair and mucous membranes by applying compositions of the present invention to the keratinous material in an amount sufficient to treat, care for and/or make up the keratinous material are provided. According to other preferred embodiments, methods of covering or hiding defects associated with keratinous material such as imperfections or discolorations by applying compositions of the present invention to the keratinous material in an amount sufficient to cover or hide such defects are provided.

According to yet other preferred embodiments, methods of enhancing the appearance of keratinous material by applying compositions of the present invention to the keratinous material in an amount sufficient to enhance the appearance of the keratinous material are provided.

In accordance with the three preceding preferred embodiments, the compositions of the present invention comprising at least one silicone resin, preferably resins and/or film formers, and glass beads are applied topically to the desired area of the skin in an amount sufficient to treat, care for and/or make up the keratinous material, to cover or hide defects associated with keratinous material, skin imperfections or discolorations, or to enhance the appearance of keratinous material. The compositions may be applied to the desired area as needed, preferably once or twice daily, more preferably once daily, and then preferably allowed to dry before subjecting to contact such as with clothing or other objects. The composition is preferably applied to the desired area that is dry or has been dried prior to application.

According to a preferred embodiment of the present invention, compositions having improved cosmetic properties such as, for example, improved long wear, transfer resistance or waterproof properties are provided. The improved properties may be chosen from improved flexibility, wearability, drying time, retention, tackiness (low), and migration over time (low).

The present invention also envisages kits and/or prepackaged materials suitable for consumer use containing one or more compositions according to the description herein. The packaging and application device for any subject of the invention may be chosen and manufactured by persons skilled in the art on the basis of their general knowledge, and adapted according to the nature of the composition to be packaged. Indeed, the type of device to be used can be in particular linked to the consistency of the composition, in particular to its viscosity; it can also depend on the nature of the constituents present in the composition, such as the presence of volatile compounds.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention.

### EXAMPLE

The following example further describes and demonstrates an embodiment within the scope of the present invention. The example is given solely for the purpose of illustration and is not to be construed as limitations of the present invention, as many variations thereof are possible without departing from the spirit and scope of the invention. All exemplified amounts are concentrations by weight of the total composition, unless otherwise specified.

| | Trade | | |
|---|---|---|---|
| Phase | Name | INCI Name | % w/w |
| A | ISOLAN | POLYGLYCERYL-4 | 1.50 |
| | GI 34 | ISOSTEARATE | |
| | ABIL | CETYL PEG/PPG-10/1 | 1.50 |
| | EM 90 | DIMETHICONE | |
| | | Pigments | 11.50 |
| | PERMETHYL | ISODODECANE | 18.00 |
| | 99A | | |
| | KP-550 | Isododecane (and) | 10.00 |
| | | ACRYLATES/ | |
| | | DIMETHICONE | |
| | | COPOLYMER | |
| | SR-1000 | TRIMETHYLSILOXY- | 6.00 |
| | | SILICATE | |
| B1 | SUNSPHERE | SILICA | 2.50 |
| | H 51 | | |
| | LUXSIL | CALCIUM SODIUM | 2.00 |
| | | BOROSILICATE VINYL | |
| | | DIMETHICONE/METH- | |
| | | ICONE SILSESQUIOXANE | |
| | | CROSSPOLYMER | |
| | KSP-100 | | 1.00 |
| B2 | Butyl- | Butylparaben | 0.20 |
| | paraben | | |
| | Bentone | Disteardimonium | 1.40 |
| | 38V | Hectorite | |
| | Propylene | Propylene | 0.70 |
| | Carbonate | Carbonate | |
| C | Water | Water | 40.30 |
| | SODIUM | SODIUM CHLORIDE | 1.00 |
| | CHLORIDE | | |
| | HYDROLITE-5 | PENTYLENE GLYCOL | 0.50 |
| | SODIUM | SODIUM | 0.20 |
| | DEHYDRO- | DEHYDROACETATE | |
| | ACETATE | | |
| | MONOHYDRATE | | |
| | Methyl- | Methylparaben | 0.20 |
| | paraben | | |
| | BRIJ 30 | Laureth-4 | 0.50 |
| | Phenoxy- | Phenoxyethanol | 0.50 |
| | ethanol | | |
| | | Total: | 100.00 |

Procedure:
1. Mix phase A ingredients, grinding with Silverson at 6000 to 7000 rpm for 30 to 40 min in an ice water bath.
2. Add phase B1 ingredients one at a time, mix well with Silverson at about 6000 rpm.
3. Add butylparaben and Benton 38V, mixing with Silverson at 6000 rpm until obtaining a good dispersion, then add propylene carbonate, mix at 6000 to 7000 rpm for 10-15 min.
4. Remove the main beaker from Silverson to Homogenizer. Warm the content to 25 to 30° C.
5. In a separate beaker, heat the phase C to 40 to 45° C. After uniform, cool the content down to 25 to 30° C.
6. Add phase C slowly to main beaker at 25 to 30° C. to form emulsion. Homogenize at 30% for 5 min.

We claim:

1. A composition comprising at least one silicone resin and glass beads.

2. The composition of claim 1, wherein the at least one silicone resin is a polymethylsilsesquioxane resin.

3. The composition of claim 1, wherein the at least one silicone resin is an MQ resin.

4. The composition of claim 1, which comprises at least two silicone resins and which are a polymethylsilsesquioxane resin and an MQ resin.

5. The composition of claim 1, wherein the at least one silicone resin is present in an amount of up to 60% by weight of the composition.

6. The composition of claim 1, wherein the at least one silicone resin is present in an amount of from 1 to 30% by weight of the composition.

7. The composition of claim 1, wherein the at least one silicone resin is present in an amount of from 1 to 15% by weight of the composition.

8. The composition of claim 1, wherein the glass beads comprise one or more borosilicates.

9. The composition of claim 1, wherein the glass beads have an average size of from about 0.5 μm to about 300 μm.

10. The composition of claim 1, which is the form of a lip compositions a foundation, or a mascara.

11. The composition of claim 1, further comprising a volatile oil.

12. The composition of claim 1, further comprising at least one pigment.

13. The composition of claim 1, which is anhydrous.

14. The composition of claim 1, further comprising water.

15. A method for treating, caring for and/or making up keratinous material comprising applying the composition of claim 1 to the keratinous material in an amount sufficient to treat, care for and/or make up the keratinous material.

16. A method for covering or hiding defects associated with keratinous material comprising applying the composition of claim 1 to the keratinous material in an amount sufficient to cover or hide such skin defects.

17. A method for enhancing the appearance of keratinous material comprising applying the composition of claim 1 to the keratinous material in an amount sufficient to enhance the appearance of the keratinous material.
